(19) United States
(12) Patent Application Publication
(10) Pub. No.: US 2024/0207335 A1
(43) Pub. Date: Jun. 27, 2024

(54) Liquid feed formulation for ruminant animals and method for preparing said liquid feed formulation

(71) Applicant: GrainCorp Limited, Sydney (AU)

(72) Inventors: Sara Labaf, Sydney (AU); Bruce Burgess, Sydney (AU)

(21) Appl. No.: 18/557,695

(22) PCT Filed: May 4, 2022
(86) PCT No.: PCT/AU2022/050415

§ 371 Date: Oct. 27, 2023

(30) Foreign Application Priority Data
May 4, 2021 (AU) 2021901313

(51) Int. Cl. A61K 36/04; A23K 10/16; A23K 10/33; A23K 20/105; A23K 20/158; A23K 20/163; A23K 50/10; A61K 9/00; A61K 31/07; A61K 31/355; A61K 47/02; A61K 47/18; A61K 47/46

(52) U.S. Cl. CPC A61K 36/04; A23K 10/16; A23K 10/33; A23K 20/105; A23K 20/158; A23K 20/163; A23K 50/10; A61K 9/0095; A61K 31/07; A61K 31/355; A61K 47/02; A61K 47/18; A61K 47/46

(57) ABSTRACT

A liquid feed formulation for reducing methane production by a ruminant animal, and a method of preparing the same are disclosed. The liquid feed formulation comprises an effective amount of at least one species of red marine microalgae suspended within a liquid nutrient, said liquid feed formulation, being pumpable, having a viscosity of between 1200 cP and 2200 cP at 23° C.

### FIELD OF THE INVENTION

The present invention relates to a liquid feed formulation for ruminant animals, and a method for preparing said liquid feed formulation.

The invention has been developed primarily for use as a liquid feed formulation to be fed to ruminant animals either directly or as part of a feed ration to reduce enteric fermentation in said ruminant animals, thereby reducing the subsequent production of the greenhouse gas (GHG), methane (CH₄), and will be described hereinafter with reference to this application.

### BACKGROUND OF THE INVENTION

Methane (CH₄) in the atmosphere is a highly a potent greenhouse gas (GHG) with a reported global warming potential 28 times greater than carbon dioxide (CO₂). Agriculture is a major contributor to the global GHG inventory and ruminant enteric fermentation is the largest agricultural source responsible for 60% of agriculture's contributions, primarily as methane.

The anti-methanogenic properties of using many types of seaweeds (macroalgae) as feed additives has now been confirmed by many researchers. Bromoform is the bioactive ingredient responsible for the anti-methanogenesis in seaweed. However, bromoform is a volatile chemical that can be easily lost to the atmosphere if not kept in a stable environment.

One particular genus of seaweed is the red marine macroalgae, Asparagopsis spp. It has been demonstrated that when Asparagopsis spp. seaweed is fed to ruminant livestock, it delivers a significant reduction in the production of methane, as well as gains in productivity.

To maintain the anti-methanogenic activity of Asparagopsis spp. seaweed, the seaweed biomass is typically processed by freeze-drying to remove nearly all of the moisture. Unfortunately, however, freeze dried Asparagopsis spp. is unpalatable to livestock.

To address this issue, it has been commonplace to add the freeze dried Asparagopsis spp. to a liquid nutrient. However, this has had a significant impact on the formulation of the liquid nutrient, affecting viscosity, which in turn affects flowability and pumpability.

More recently, it has been demonstrated that an alternative process to freeze-drying involves homogenising freshly collected Asparagopsis spp. in oil. However, the inability to keep the oil fully dispersed has so far prevented this process from being used commercially for producing liquid feed formulations for ruminant animals.

The present invention thus seeks to provide a liquid feed formulation for ruminant animals, and a method for preparing said liquid feed formulation, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the present invention there is provided a liquid feed formulation for reducing methane production by a ruminant animal, said liquid feed formulation comprising an effective amount of at least one species of red marine microalgae suspended within a liquid nutrient, said liquid feed formulation, being pumpable, having a viscosity of between 1200 cP and 2200 cP at 23° C.

Preferably, the species of red marine microalgae is an Asparagopsis spp. selected from the group consisting of Asparagopsis taxiformis and Asparagopsis armata.

In one embodiment, the liquid feed formulation has a dry matter content of from about 50% to about 75% by weight.

In one embodiment, the species of red marine microalgae is freeze dried.

In one embodiment, the species of red marine microalgae is homogenised in an edible oil selected from the group consisting of canola oil, sunflower oil, safflower oil, soybean oil, and any combination thereof.

In one embodiment, the species of red marine microalgae comprises an anti-methanogenic agent.

Suitably, the anti-methanogenic agent is bromoform.

In one embodiment, the bromoform is present in the liquid feed formulation in an amount from about 100 mg/kg to about 700 mg/kg by weight.

Preferably, the liquid nutrient comprises water.

In one embodiment, the water is present in the liquid feed formulation in an amount from about 10% to about 35% by weight.

Preferably, the liquid nutrient comprises a carbohydrate source.

In one embodiment, the carbohydrate source is present in the liquid feed formulation in an amount of about 20% to about 80% by weight.

More preferably, the carbohydrate source comprises a sugar-based carbohydrate source.

In one embodiment, the sugar-based carbohydrate source comprises molasses and/or glycerine.

Preferably, the liquid nutrient comprises an edible oil selected from the group consisting of canola oil, sunflower oil, safflower oil, soybean oil, and any combination thereof.

In one embodiment, the edible oil is present in the liquid feed formulation in an amount from about 0.01% to about 0.5% by weight. In one embodiment, the edible oil comprises canola oil.

Preferably, the liquid nutrient comprises a thickener selected from the group consisting of xanthan gum, sodium alginate, gum arabic, sodium carboxymethylcellulose, guar gum, maltodextrin, pregelatinized starch and soy protein binder.

In one embodiment, the thickener is present in the liquid feed formulation in an amount from about 0.01% to about 0.5% by weight.

In one embodiment, the thickener comprises xanthan gum.

Preferably, the liquid feed formulation further comprises one or more excipients or additives selected from the group consisting of wetting agents, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen).

In one embodiment, the effective amount of the at least one species of red marine microalgae suspended within the liquid nutrient is from about 2% to about 15% by weight.

Preferably, the liquid nutrient comprises:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source.

In one embodiment, the edible oil is canola oil, the carbohydrate source is molasses, and the thickener is xanthan gum.

According to a second aspect of the present invention there is provided a liquid feed formulation for reducing methane production by a ruminant animal, said liquid feed formulation comprising an effective amount of at least one species of red marine microalgae suspended within a liquid nutrient comprising:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source, wherein the liquid feed formulation is pumpable, having a viscosity of between 1200 cP and 2200 cP at 23° C.

In one embodiment, the species of red marine microalgae is Asparagopsis taxiformis.

In one embodiment, the effective amount of Asparagopsis taxiformis suspended within the liquid nutrient is from about 2% to about 15% by weight.

Suitably, the Asparagopsis taxiformis comprises an anti-methanogenic agent in the form of bromoform.

In one embodiment, the bromoform is present in the liquid feed formulation in an amount from about 100 mg/kg to about 700 mg/kg by weight.

In one embodiment, the liquid feed formulation has a dry matter content of from about 50% to about 75% by weight.

According to a third aspect of the present invention there is provided a method of feeding a ruminant animal, the method comprising feeding the liquid feed formulation of the first or second aspect to a ruminant animal.

Preferably, the liquid feed formulation is mixed with animal feed.

In one embodiment, the liquid feed formulation is mixed with the animal feed at a ratio of about 2% to about 8% relative to the combined weight of the liquid feed formulation and the animal feed.

In one embodiment, the animal feed comprises a feedstock selected from the group consisting of dry animal fodder, straw, hay, alfalfa, grains, forage, grass, fruits, vegetables, oats, crop residue, fats and oils and any combination thereof.

Preferably, said ruminant animal is cattle, sheep or goat.

According to a fourth aspect of the present invention there is provided a method for reducing methane production by a ruminant animal, said method comprising the step of administering to said animal a liquid feed formulation comprising an effective amount of at least one species of red marine microalgae suspended within a liquid nutrient, said liquid feed formulation being pumpable, having a viscosity of between 1200 cP and 2200 cP at 23° C.

Preferably, the method further comprises the step of mixing the liquid feed formulation with animal feed.

In one embodiment, the liquid feed formulation is mixed with the animal feed at a ratio of about 2% to about 8% relative to the combined weight of the liquid feed formulation and the animal feed.

In one embodiment, the animal feed comprises a feedstock selected from the group consisting of dry animal fodder, straw, hay, alfalfa, grains, forage, grass, fruits, vegetables, oats, crop residue, fats and oils and any combination thereof.

Preferably, said ruminant animal is cattle, sheep or goat.

According to a fifth aspect of the present invention there is provided a method of producing a liquid feed formulation for reducing methane production by a ruminant animal, the method comprising the step of suspending an effective amount of at least one species of red marine microalgae within a liquid nutrient to form a liquid feed formulation that is pumpable, having a viscosity of between 1200 cP and 2200 cP at 23° C.

Preferably, the species of red marine microalgae is an Asparagopsis spp. selected from the group consisting of Asparagopsis taxiformis and Asparagopsis armata.

In one embodiment, the liquid feed formulation has a dry matter content of from about 50% to about 75% by weight

In one embodiment, the species of red marine microalgae is freeze dried.

Preferably, the liquid nutrient comprises:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source.

In one embodiment, the water, thickener and edible oil of the liquid nutrient are combined sequentially, prior to suspending the at least one species of red marine microalgae therewithin.

Preferably, the carbohydrate source is introduced to the liquid nutrient after the at least one species of red marine microalgae has been suspended therewithin.

In one embodiment, prior to suspending the species of red marine microalgae within the liquid nutrient, the method comprises the step of homogenising said species of red marine microalgae in an edible oil selected from the group consisting of canola oil, sunflower oil, safflower oil, soybean oil, and any combination thereof.

Preferably, the liquid nutrient comprises:
(a) about 10% to about 35% by weight of water;
(b) about 0.01% to about 0.5% by weight of a thickener; and
(c) about 20% to about 80% by weight of a carbohydrate source.

Preferably, the water and thickener of the liquid nutrient are combined sequentially, prior to suspending the homogenised species of red marine microalgae therewithin.

Preferably, the carbohydrate source is introduced to the liquid nutrient after the homogenised species of red marine microalgae has been suspended therewithin.

In one embodiment, the species of red marine microalgae comprises an anti-methanogenic agent.

Suitably, the anti-methanogenic agent is bromoform.

In one embodiment, the bromoform is present in the liquid feed formulation in an amount from about 100 mg/kg to about 700 mg/kg by weight.

In one embodiment, the method further comprises the step of introducing one or more excipients or additives to the liquid nutrient prior to suspending the at least one species of red marine microalgae therewithin.

Preferably, the one or more excipients or additives are selected from the group consisting of wetting agents, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen).

According to a sixth aspect of the present invention there is provided a liquid feed formulation for reducing methane production by a ruminant animal, the liquid feed formulation prepared according to the method of the fifth aspect.

Other aspects of the invention are also disclosed.

cl

It should be noted in the following description that like or the same reference numerals in different embodiments denote the same or similar features.

The present invention is predicated on the finding that Asparagopsis spp., whether it be freeze-dried or fully immersed in oil, can be incorporated into a molasses base liquid nutrient to render it palatable and acceptable to livestock.

This molasses based liquid feed formulation can be fed to ruminant animals in various forms, either as part of a mixed feed ration or directly to the animal. In either form, the liquid feed formulation supplements the animal's requirements for nutrients such as energy, protein, trace minerals and vitamins, as well as delivering various medications. Those fed directly to ruminant animals can be formulated to varying levels of palatability to control intake. Those mixed in a feed ration improve the palatability of the ration, reduce dust and separation, as well as providing an even distribution of the nutrients/components supplied. The liquid feed formulation contains both soluble and insoluble components that require enough available moisture to dissolve the soluble components, while maintaining a sufficient level of viscosity to suspend the insoluble components.

In addition, the inventors have successfully demonstrated that the liquid feed formulation is flowable, pumpable, and able to be stored for a period of time, while maintaining good consistency without the components settling out or separating from the liquid feed formulation.

In this respect, combining Asparagopsis spp. with a molasses-based liquid nutrient enables the delivery of the Asparagopsis spp. seaweed to ruminant animals in a stable, palatable form, utilizing an existing supply chain, while maintaining a sufficient amount of bromoform within the Asparagopsis spp. to facilitate the anti-methanogenesis activity.

### Definitions

As used herein, the term “reducing” includes the reduction of amount of substance in comparison with a reference. For example, the reduction in the amount of total methane produced by a ruminant animal or animals administered a liquid feed formulation comprising a red marine macroalgae according to the present invention, relative to an animal or animals not administered the liquid feed formulation of the present invention. The reduction can be measured in vitro with an artificial rumen system that simulates anaerobic fermentation, or in vivo with animals confined in respiration chambers. It is within the knowledge and skill of those trained in the art to assess enteric methanogenesis by a ruminant animal.

As used herein, the term “reducing methane production” refers to the reduction of methane produced in the animal's digestive system. The term includes the specific volume of methane generated as a result of anaerobic fermentation, for example, in the systems described herein. Fermentation in the rumen and the gut of a ruminant gives rise to production of methane. The present invention aims to reduce this process, such as to reduce the total amount of methane produced in the animal's digestive system. It is within the knowledge and skill of those trained in the art to assess methane production by a ruminant animal.

As used herein, the term “effective amount”, is used to indicate a quantity of at least one species of red marine macroalgae sufficient to allow improvement, e.g. reduction in the amount of methane production in comparison with a reference or control. Within the meaning of the present invention, the methane reductive effect can be measured in the rumen with an artificial rumen system, or by in vivo oral administration to ruminants.

As used herein, the terms “administer” and “administered”, are used to indicate the action of introducing at least one species of red marine macroalgae according to the present invention into the ruminant animal's digestive system. More particularly, this administration is an administration by oral route. This administration can in particular be carried out by supplementing a feed ration intended for the ruminant animal with the liquid feed formulation of the present invention, the thus supplemented feed ration then being ingested by the ruminant animal. The administration can also be carried out using a stomach tube or any other means making it possible to directly introduce the red marine macroalgae into the animal's digestive system.

As used herein, the term “ruminant”, is used to indicate a herbivorous mammal of the suborder Ruminantia; animals that are able to acquire nutrients from plant-based food by fermenting it in a specialized stomach prior to digestion, principally through microbial action. For the purpose of describing the present invention, said ruminant animal is cattle, sheep or goat.

As used herein, the term “homogenising”, is used to indicate the breakup the red marine microalgae biomass to facilitate release of at least one anti-methanogenic agent from the biomass. The homogenising can take place by, for example, crushing, grinding, milling, blending, cutting, slicing, dicing, or any combination thereof.

As used herein, the term “shelf life” is used to indicate that the amount or level of an anti-methanogenic agent in the liquid feed formulation according to the embodiments of the present invention is not significantly reduced for the period of time and temperature that the liquid feed formulation is stored at. In other words, the amount of time elapsed before there is a significant reduction in the level of the anti-methanogenic agent is taken to indicate the shelf life. The term “stability” is also used as an indicator of shelf life. The ‘stability’ of the anti-methanogenic agent can be assessed by measuring the level of the anti-methanogenic agent over time.

As used herein, the term “pumpability” is used to indicate the ability of the liquid feed formulation of the present invention to be pumped for the purpose of transportation, transferring to storage tanks, transferring to mixing plants, mixing in feed or transferring to feed troughs.

As used herein, the term “flowability” is used to indicate the capacity of the liquid feed formulation of the present invention to flow for the purpose of transportation, transferring to storage tanks, transferring to mixing plants, mixing in feed or transferring to feed troughs.

As used herein, “dry matter (DM) content” is used to indicate the percent of the liquid feed formulation of the present invention that is not water, assuming that all the moisture from the liquid feed formulation has been removed.

As used herein, “as-fed” is used to indicate the percent of the liquid feed formulation of the present invention in which none of the moisture has been removed.

### Liquid Feed Formulation

A liquid feed formulation for reducing methane production by a ruminant animal according to a preferred embodiment of the present invention, will now be described.

There are two recognised species of Asparagopsis present throughout the world, one is a tropical/sub-tropical (Asparagopsis taxiformis) type and the other is a temperate (Asparagopsis armata) type. In either case, both species comprise an anti-methanogenic agent in the form of bromoform. It is this agent that is responsible for reducing enteric fermentation in ruminant animals.

According to the embodiments of the present invention described herein, the Asparagopsis species used in the preparation of the liquid feed formulations is Asparagopsis taxiformis.

However, it will be appreciated by persons skilled in the relevant art that the liquid feed formulations according to the present invention may also be prepared using Asparagopsis armata.

Asparagopsis taxiformis and Asparagopsis armata are harvested from the sea as a biomass. Once harvested, these species are then typically processed either by freeze drying or by immersing in oil in order to retain as much of the anti-methanogenic agent, bromoform as possible.

What follows therefore is a description of two methods for preparing liquid feed formulations for ruminant animals in which the Asparagopsis taxiformis is provided in either a freeze-dried form or an oil-immersed form.

### Freeze Dried Asparagopsis

The Asparagopsis taxiformis biomass is freeze dried according to standard freeze-drying procedures.

The quantity of bromoform in the freeze-dried Asparagopsis taxiformis is typically between about 2 and about 40 mg/g according to the dry matter (DM) weight.

According to one embodiment of the present invention, the bromoform level in the freeze-dried Asparagopsis taxiformis used to prepare the liquid feed formulation of Example 1 (see Table 1) was calculated to be 6.5 mg/g DM.

In this embodiment, the liquid feed formulation is prepared by suspending the freeze-dried Asparagopsis taxiformis in a liquid nutrient that comprises in its basic form, water, a thickener, a carbohydrate source, and an edible oil.

The carbohydrate source is a sugar-based carbohydrate source selected from the group consisting of molasses and glycerine.

The thickener is selected from the group consisting of xanthan gum, sodium alginate, gum arabic, sodium carboxymethylcellulose, guar gum, maltodextrin, pregelatinized starch and soy protein binder.

The edible oil is a vegetable oil selected from the group consisting of canola oil, sunflower oil, safflower oil, soybean oil, and any combination thereof.

According to this embodiment, the liquid nutrient for suspending the freeze-dried Asparagopsis taxiformis in, comprises from:
(a) about 10% to about 35% by weight of water;
(b) about 0.01% to about 0.5% by weight of xanthan gum;
(c) about 0.01% to about 0.5% by weight of canola oil; and
(d) about 20% to about 80% by weight of molasses.

The inventors have found that it is preferable to the liquid feed formulation that these components are added in the order of addition in which they appear in Table 1 (see below), and that the mixing time (minutes) between certain additions is sufficient to allow the soluble components of the liquid nutrient to be fully dissolved before adding the freeze-dried Asparagopsis taxiformis.

In particular, the inventors have found that it is critical that the canola oil and xanthan gum of the liquid nutrient are pre-batched then added to water first, to enable full hydration of the xanthan gum.

It is also critical that once the xanthan gum is fully hydrated, the subsequent components from ammonium sulfate through to vitamin E 75% are then added in the order of their solubility in water (as listed in Table 1) to ensure that they are fully dissolved in solution before the highly moisture absorbent freeze-dried Asparagopsis taxiformis is added.

Once the freeze-dried Asparagopsis taxiformis has been suspended within the liquid nutrient, the carbohydrate source (molasses) is then introduced into the liquid nutrient in order to optimize the viscosity of the liquid feed formulation.

The inventors have determined that for a base formulation (that is a liquid feed formulation with 0% Asparagopsis taxiformis added) having a dry matter (DM) content of 68-70%, every 1% of Asparagopsis taxiformis added as part of the liquid feed formulation, an additional 2% water needs to be added, replacing an equivalent percentage of the carbohydrate source (molasses).

In this embodiment, the effective amount of freeze-dried Asparagopsis taxiformis suspended within the liquid nutrient is determined to be from about 2% to about 15% by weight. This would equate to a bromoform content in the resulting liquid feed formulation, measured immediately after preparation, of from about 100 mg/kg to about 700 mg/kg by weight.

The inventors have determined that the liquid feed formulation in Table 1 has a formulated bromoform level of 455 mg/kg (as fed). It will be understood by persons of skill in the relevant art that this liquid feed formulation, when included in a feed ration at 5%, will provide 22.75 mg/kg (as fed) or 25.85 mg/kg (Dry Matter for a feed ration with a dry matter content of 88%) of bromoform in the finished feed ration.

The liquid feed formulation prepared according to this embodiment has an average dry matter content of from about 50% to about 75% by weight, resulting in a viscosity of between 1200 cP and 2200 cP at 23° C.

Viscosity measurements are used for two key purposes.
a) To achieve a consistency that will allow insoluble components to remain in suspension. The inventors have found that the lower desired limit of 1200 cP is the point that insoluble components become susceptible to “falling out” or settling.
b) To measure pumpability and flowability. Viscosity is directly related to those characteristics. The inventors have found that the upper desired limit of 2200 cP is the level that pumpability and flowability start to be compromised, such that the liquid feed formulation becomes too thick to pump or flow efficiently.

By virtue of falling within this particular viscosity range, the liquid feed formulation is efficiently flowable and pumpable, thereby rendering it suitable for the purpose of transportation, transferring to storage tanks, transferring to mixing plants, mixing in feed or transferring to feed troughs.

### Excipients/Additives

According to some embodiments, the liquid feed formulation of the present invention may further comprise one or more excipients that are necessary to prepare the desired feed form and/or one or more additives aimed at improving the quality of the liquid feed formulation.

The one or more excipients or additives may be selected from the group consisting of wetting agents, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen).

In a preferred embodiment, the one or more excipients or additives are introduced successively into the liquid nutrient in the order of addition in which they appear in Table 1 (see below), prior to suspending the freeze-dried Asparagopsis taxiformis therewithin.

### EXAMPLES

What follows is a liquid feed formulation comprising freeze-dried Asparagopsis taxiformis for use as part of a mixed feed ration for a ruminant animal.

### Example 1

### Preparation of a Liquid Feed Formulation Comprising Freeze-Dried Asparagopsis taxiformis

TABLE 1

| Component | % By Weight | Mixing Time (min) |
|---|---|---|
| Water | 30.0 | 0 |
| Xanthan gum | 0.04 | 0 |
| Canola oil | 0.1 | 10 |
| Ammonium sulfate | 2.7 | 5 |
| Urea | 6.9 | 5 |
| Salt (fine dried) | 3.3 | 0 |
| Trace Mineral Premix^{a} | 0.5 | 0 |
| Copper 12.7%^{b} | 0.025 | 0 |
| Monensin 400^{c} | 0.11 | 5 |
| Magnesium carbonate | 2.0 | 0 |
| Vitamin A 40%^{d} | 0.02 | 0 |
| Vitamin E 75%^{e} | 0.0267 | 5 |
| Asparagopsis taxiformis | 7.0 | 10 |
| Molasses | 47.2783 | 10 |

a—The trace mineral premix (manufactured and supplied by Rabar Pty Ltd) comprises zinc (12%), manganese (4%), cobalt (0.177%), iodine (1.178%), selenium (0.265%) and salt (51.17%).
b—SQM™ Copper (manufactured and supplied by Qualitech, Inc.).
c—Monensin 400 (manufactured and supplied by Nutriment Health, Phibro Animal Health or International Animal Health).
d—Rovimix™ A 400 DLC (manufactured and supplied by DSM Animal Nutrition & Health).
e—Rovimix™ E 750 DLC Liquid (manufactured and supplied by DSM Animal Nutrition & Health).

What follows is a liquid feed formulation comprising freeze-dried Asparagopsis taxiformis for directly feeding to a ruminant animal.

### Example 2

### Preparation of a Liquid Feed Formulation Comprising Freeze-Dried Asparagopsis taxiformis

TABLE 2

| Component | % By Weight | Mixing Time (min) |
|---|---|---|
| Water | 23.2 | 0 |
| Xanthan gum | 0.04 | 0 |
| Canola oil | 0.05 | 10 |
| Ammonium sulfate | 5.0 | 5 |
| Aluminium sulfate | 0.5 | 5 |
| Urea | 2.0 | 5 |
| Salt (fine dried) | 2.5 | 5 |
| Trace Mineral Premix^{a} | 0.25 | 0 |
| Copper 12.7%^{b} | 0.05 | 5 |
| Asparagopsis taxiformis | 4.2 | 10 |
| Molasses | 60.515 | 0 |
| Phosphoric acid 81% | 1.74 | 10 |

^{a}The trace mineral premix (manufactured and supplied by Rabar Pty Ltd) comprises zinc (12%), manganese (4%), cobalt (0.177%), iodine (1.178%), selenium (0.265 %) and salt (51.17%).
^{b}SQM ™ Copper (manufactured and supplied by Qualitech, Inc.).

### Oil Immersed Asparagopsis

The Asparagopsis taxiformis biomass is homogenised in an edible oil according to the method outlined in the literature [M. Magnusson et al. Algal Research, 51 (2020) 102065]

In this embodiment, the edible oil is canola oil. Other vegetable oils suitable for this purpose may also include sunflower oil, safflower oil, soybean oil, and any combination thereof.

The quantity of bromoform in the oil immersed Asparagopsis taxiformis is calculated to be between about 2 mg/g and about 40 mg/g according to the dry matter (DM) weight.

Once homogenised, the resulting oil immersed Asparagopsis taxiformis is then suspended in a liquid nutrient that comprises in its basic form, water, a thickener, and a carbohydrate source.

The carbohydrate source is a sugar-based carbohydrate source selected from the group consisting of molasses and glycerine.

The thickener is selected from the group consisting of xanthan gum, sodium alginate, gum arabic, sodium carboxymethylcellulose, guar gum, maltodextrin, pregelatinized starch and soy protein binder.

In a preferred embodiment, the liquid nutrient for suspending the oil immersed Asparagopsis taxiformis in, comprises from:
(a) about 10% to about 35% by weight of water;
(b) about 0.01% to about 0.5% by weight of xanthan gum; and
(c) about 20% to about 80% by weight of molasses.

Again, the inventors have found that it is preferable that these components are added in the order of addition in which they appear in Table 3 and Table 4 (see below), and that the mixing time (minutes) between certain additions is sufficient to allow the soluble components of the liquid nutrient to be fully dissolved before adding the oil immersed Asparagopsis taxiformis.

For instance, the water and thickener (xanthan gum) of the liquid nutrient are combined sequentially, prior to suspending the oil immersed Asparagopsis taxiformis therewithin.

The carbohydrate source (molasses) is then introduced into the liquid nutrient after the oil immersed Asparagopsis taxiformis has been suspended therewithin.

In this embodiment, the effective amount of oil immersed Asparagopsis taxiformis suspended within the liquid nutrient is from about 2% to about 15% by weight. This equates to a bromoform content in the resulting liquid feed formulation, measured immediately after preparation, of from about 100 mg/kg to about 700 mg/kg by weight.

The liquid feed formulation prepared according to this embodiment has an average dry matter content of from about 50% to about 75% by weight, resulting in a viscosity of between 1200 cP and 2200 cP at 23° C.

In much the same way as the liquid feed formulations comprising the freeze-dried Asparagopsis taxiformis described above, the liquid feed formulation comprising oil immersed Asparagopsis taxiformis may also comprise one or more of the same excipients and/or additives described above to improve the quality of the liquid feed formulation.

What follows is a liquid feed formulation comprising oil immersed Asparagopsis taxiformis for use as part of a mixed feed ration for a ruminant animal.

### Example 3

### Preparation of a Liquid Feed Formulation Comprising Oil Immersed Asparagopsis taxiformis

TABLE 3

| Component | % By Weight | Mixing Time (min) |
|---|---|---|
| Water | 16.0 | 0 |
| Emulsifier^{a} | 0.25 | 0 |
| Xanthan gum | 0.1 | 0 |
| Asparagopsis taxiformis^{b} | 11.0 | 10 |
| Ammonium sulfate | 2.7 | 5 |
| Urea | 6.9 | 5 |
| Salt (fine dried) | 3.3 | 0 |
| Trace Mineral Premix^{c} | 0.5 | 0 |
| Copper 12.7%^{d} | 0.025 | 0 |
| Monensin 400^{e} | 0.11 | 5 |
| Magnesium carbonate | 2.0 | 0 |
| Vitamin A 40%^{f} | 0.02 | 0 |
| Vitamin E 75%^{g} | 0.0267 | 5 |
| Molasses | 57.0683 | 10 |

^{a}The emulsifier for dispersing the oil is KEM WET OZ ™ (manufactured and supplied by Kemin Industries, Inc.).
^{b}The immersion oil is canola oil.
^{c}The trace mineral premix (manufactured and supplied by Rabar Pty Ltd) comprises zinc (12%), manganese (4%), cobalt (0.177%), iodine (1.178%), selenium (0.265 %) and salt (51.17%).
^{d}SQM ™ Copper (manufactured and supplied by Qualitech, Inc.).
^{e}Monensin 400 (manufactured and supplied by Nutriment Health, Phibro Animal Health or International Animal Health).
^{f}Rovimix ™ A 400 DLC (manufactured and supplied by DSM Animal Nutrition & Health).
^{g}Rovimix ™ E 750 DLC Liquid (manufactured and supplied by DSM Animal Nutrition & Health).

What follows is another liquid feed formulation comprising oil immersed Asparagopsis taxiformis for use as part of a mixed feed ration for a ruminant animal, this time comprising limestone (CaCO₃).

### Example 4

### Preparation of a Liquid Feed Formulation Comprising Oil Immersed Asparagopsis taxiformis

TABLE 4

| Component | % By Weight | Mixing Time (min) |
|---|---|---|
| Water | 21.5 | 0 |
| Emulsifier^{a} | 0.25 | 0 |
| Xanthan gum | 0.12 | 0 |
| Asparagopsis taxiformis^{b} | 11.0 | 10 |
| Ammonium sulfate | 3.5 | 5 |
| Urea | 6.9 | 5 |
| Salt (fine dried) | 3.3 | 0 |
| Trace Mineral Premix^{c} | 0.5 | 0 |
| Copper 12.7%^{d} | 0.025 | 0 |
| Monensin 400^{e} | 0.11 | 5 |
| Magnesium carbonate | 2.0 | 0 |
| Vitamin A 40%^{f} | 0.02 | 0 |
| Vitamin E 75%^{g} | 0.0267 | 5 |
| Molasses | 23.7483 | 10 |
| Limestone (fine) | 27.0 | 10 |

^{a}The emulsifier for dispersing the oil is KEM WET OZ ™ (manufactured and supplied by Kemin Industries, Inc.).
^{b}The immersion oil is canola oil.
^{c}The trace mineral premix (manufactured and supplied by Rabar Pty Ltd) comprises zinc (12%), manganese (4%), cobalt (0.177%), iodine (1.178%), selenium (0.265 %) and salt (51.17%).
^{d}SQM ™ Copper (manufactured and supplied by Qualitech, Inc.).
^{e}Monensin 400 (manufactured and supplied by Nutriment Health, Phibro Animal Health or International Animal Health).
^{f}Rovimix ™ A 400 DLC (manufactured and supplied by DSM Animal Nutrition & Health).
^{g}Rovimix ™ E 750 DLC Liquid (manufactured and supplied by DSM Animal Nutrition & Health).

What follows is a liquid feed formulation comprising oil immersed Asparagopsis taxiformis for directly feeding to a ruminant animal.

### Example 5

### Preparation of a Liquid Feed Formulation Comprising Oil Immersed Asparagopsis taxiformis

TABLE 5

| Component | % By Weight | Mixing Time (min) |
|---|---|---|
| Water | 14.0 | 0 |
| Emulsifierª | 0.15 | 0 |
| Xanthan gum | 0.1 | 0 |
| Asparagopsis taxiformis^{b} | 6.8 | 10 |
| Ammonium sulfate | 5.0 | 5 |
| Aluminium sulfate | 0.5 | 5 |
| Urea | 2.1 | 5 |
| Salt (fine dried) | 2.5 | 5 |
| Trace Mineral Premix^{c} | 0.25 | 0 |
| Copper 12.7%^{d} | 0.005 | 5 |
| Molasses | 66.855 | 0 |
| Phosphoric acid 81% | 1.74 | 10 |

^{a}The emulsifier for dispersing the oil is KEM WET OZ ™ (manufactured and supplied by Kemin Industries, Inc.).
^{b}The immersion oil is canola oil.
^{c}The trace mineral premix (manufactured and supplied by Rabar Pty Ltd) comprises zinc (12%), manganese (4%), cobalt (0.177%), iodine (1.178%), selenium (0.265 %) and salt (51.17%).
^{d}SQM ™ Copper (manufactured and supplied by Qualitech, Inc.).

### Feed Ration

The molasses based liquid feed formulations prepared according to embodiments of the present invention can be fed to ruminant animals in various forms, either as part of a mixed feed ration or directly to the animal.

In either form, the liquid feed formulation supplements the animal's requirements for nutrients such as energy, protein, trace minerals and vitamins, as well as delivering various medications. Those fed directly to ruminant animals can be formulated to varying levels of palatability to control intake. Those mixed in a feed ration improve the palatability of the ration, reduce dust and separation, as well as providing an even distribution of the nutrients/components supplied.

What follows is a description of a method of preparing a feed ration based on the liquid feed formulation of the embodiments of the present invention in combination with a feedstock for use in feeding to a ruminant animal such as cattle, sheep or goat.

The feedstock may be selected from the group consisting of dry animal fodder, straw, hay, alfalfa, grains such as wheat, barley, sorghum wheat), roughage (hay, straw, silage), protein meals (cotton seed meal, canola meal), high protein seeds (cotton seed, lupins, chickpeas), forage, grass, fruits, vegetables, oats, crop residue, fats and oils and any combination thereof.

In one embodiment, the liquid feed formulation is mixed with the feedstock at a ratio of about 2% to about 8% relative to the combined weight of the liquid feed formulation and the feedstock.

### Example 6

### Preparation of a Feed Ration

A typical feed ration for feeding to a ruminant animal to reduce methane production comprises: 0% to 90% grain (wheat, barley, sorghum etc.), 2% to 95% roughage (hay, straw, silage), 0% to 12% protein meals (cotton seed meal, canola meal), 0% to 12% high protein seeds (cotton seed, lupins, chickpeas), 0% to 4% oil and a 2% to 8% concentration of any one of the liquid feed formulations of the present invention described above.

What follows is a sample starter feed ration based on the liquid feed formulation described in Example 1 (see Table 1).

TABLE 6
Starter Feed Ration

| Component | % By Weight |
|---|---|
| Oaten hay | 30.8 |
| Sorghum silage | 20.0 |
| Barley 12.0 | 20.0 |
| Wheat 12.0 | 10.0 |
| Cereal straw | 6.0 |
| Liquid Feed Formulation (Example 1) | 4.2 |
| Canola meal 38.0 | 4.0 |
| Cotton seed | 4.0 |
| Limestone (fine) | 1.0 |

The starter feed ration in Table 6 provides a formulated bromoform level of 19.11 mg/kg (as fed) or 25.36 mg/Kg (Dry Matter for a feed ration with a dry matter content of 75.35%). This equates to the high level used in the Asparagopsis feedlot feeding trial conducted by the Commonwealth Scientific and Industrial Research Organisation (CSIRO) on behalf of the Meat & Livestock Australia Limited (Published: 31 May 2018 (see: https://www.mla.com.au/research-and-development/reports/2020/asparagopsis-feedlot-feeding-trial/).

What follows is a sample finisher feed ration based on the liquid feed formulation described in Example 1 (see Table 1).

TABLE 7
Finisher Feed Ration

| Component | % By Weight |
|---|---|
| Barley 12.0 | 43.2 |
| Sorghum silage | 20.0 |
| Wheat 12.0 | 20.0 |
| Cereal straw | 6.0 |
| Liquid Feed Formulation (Example 1) | 4.3 |
| Cotton seed | 4.0 |
| Vegetable oil (blend) | 1.5 |
| Limestone (fine) | 1.0 |

The finisher feed ration in Table 7 provides a formulated bromoform level of 19.57 mg/kg (as fed) or 25.62 mg/Kg (Dry Matter for a feed ration with a dry matter content of 76.35%). This also equates to the high level used in the CSIRO trial.

### MATERIALS AND METHODS

As described above, the components of each liquid feed formulation are added in the order of addition in which they appear in the corresponding table (see Tables 1-5).

By way of a guide, the following method outlines the procedure for preparing the liquid feed formulation described in Example 1 (see Table 1), for every 1000 kg's mixed.

Firstly, 0.4 Kg of xanthan gum and 1.0 Kg of canola oil are placed in the vessel of a vertical shear feed mixer (custom built) and 300 Kg of water is added, and the mixture stirred at a stirring rate of 800 to 850 rpm for around 10 minutes to enable full hydration of the xanthan gum.

Once the xanthan gum is fully hydrated, the following components, ammonium sulfate (27 Kg), urea (69 Kg), fine dried NaCl (33 Kg), Tracer Mineral Premix™ (5.0 Kg), Copper 12.7% (0.25 Kg), Monensin 400 (1.1 Kg), MgCO₃ (20.0 Kg), Vitamin A 40% (0.2 Kg) and Vitamin E 75% (0.267 Kg), are then added to the vessel one by one with gradual stirring at a stirring rate of 700 to 750 rpm for a total time of around 20 minutes.

Next, 70 Kg of freeze-dried Asparagopsis taxiformis is then added to the vessel, and then stirred at a speed of 700 to 750 rpm for around 10 minutes to hydrate and suspend the Asparagopsis taxiformis therein.

Lastly, 472.783 Kg of molasses is then gradually introduced into the vessel at a stirring rate of 700 to 750 rpm for around 10 minutes to obtain the liquid feed formulation.

The viscosity of the liquid feed formulation is measured using a Brookfield Viscometer (supplied by the John Morris Group), the moisture content (%) and the dry matter (DM) content were determined by the formulation specifications obtained using the Format ARA formulations program (supplied by Cargill Format Solutions).

The bromoform content within the Asparagopsis taxiformis starting materials, and within the liquid feed formulation itself, is determined by gas chromatography-mass spectrometry (GC-MS) using methanol extraction.

For the freeze-dried samples, bromoform was extracted by sonicating 100 mg of homogenized seaweed (using a pestle and mortar) with 10 mL of high-performance liquid chromatography-grade methanol, for 30 minutes at 4° C. The sample was then centrifuged at 3000g for 10 minutes, and the methanol removed and transferred into a clean 50mL Falcon tube. The extraction process was repeated using 10 mL of methanol for 30 minutes at 4° C. in a sonication bath. After centrifugation, a further 20 mL methanol was added to dilute the sample. The methanol phase was then collected and analysed by GC-MS as described below.

Gas chromatography was performed using an Agilent gas chromatograph 8890 (supplied by Agilent, Australia) equipped with a polyethylene glycol-coated phase on a polyimide-coated fused silica capillary column (INNOWAX 19091N1331, 30 m, 0.25 mm i.d., 0.25 μM). All injections (1 μl) were performed in the splitless mode. The inlet liner (Agilent 5190-2293, splitless, single taper, glass wool) was replaced after 50 samples. The injection port was held at 180° C. and the GC-MS interface at 280° C. The GC was held at 40° C. for 1 minute, ramped at 16° C. min⁻¹ to 250° C., then held at this temperature for 2 minutes. Helium was used as the carrier gas. Mass spectrometry was performed on an Agilent 7010 triple quadrupole mass spectrometer (supplied by Agilent, Australia). Ions characteristic of the standards and target compounds were monitored in the multiple reaction monitoring (MRM) mode. Target compound standards were run at the start of each sample set and at regular intervals within sample sets. The target compound concentration in each sample was calculated from the peak area. This area was then converted to concentration by reference to standard curves. Masses of target compounds are expressed as mass compound per unit algal dry weight. Commercial standards used were bromoform (Sigma-Aldrich).

### RESULTS

### Flowability/Pumpability

The inventors calculated the viscosity of the liquid feed formulations of Examples 1 to 5 over a period of about 3 months to map their degradation over time.

Viscosity data (cP) obtained for the liquid feed formulations of Examples 1 to 5, taken (23° C.) at 0 weeks, 1 week and 8 weeks after the date of manufacture is provided in Table 8.

TABLE 8
Viscosity Data for the liquid feed formulations

| | | Viscosity (cP) | | | | |
|---|---|---|---|---|---|---|
| Date | | Ex. 1 | Ex. 2 | Ex. 3 | Ex. 4 | Ex. 5 |
| 0 | weeks | 1030 | 1340 | 1370 | 1350 | 1470 |
| 1 | week | 1770 | 1810 | 1610 | 1660 | 1720 |
| 8 | weeks | 2180 | 2060 | 1830 | 1930 | 1990 |

As shown in Table 8, the viscosity (taken at 23° C.) of the liquid feed formulation of Example 1 (“Ex. 1”) increased on standing to a final value of 2180 cP, which is still within the acceptable viscosity range of between 1200 cP and 2200 cP for the purpose of transportation, transferring to storage tanks, transferring to mixing plants, mixing in feed, or transferring to feed troughs.

Similar viscosity readings were observed for the liquid feed formulations of Examples 2 to 5.

### CONCLUSION

The inventors have prepared liquid feed formulations comprising Asparagopsis species seaweed in either a freeze-dried or oil immersed form, for feeding to ruminant animals, in which the liquid feed formulations have a viscosity that allows it to flow or be pumped efficiently for effective transportation, transferring to storage tanks, transferring to mixing plants, mixing in feed or transferring to feed troughs. The inventors have also determined that the as-prepared liquid feed formulations help to minimise the loss of bromoform associated with the Asparagopsis species to facilitate the desired anti-methanogenesis activity for reducing the amount of methane produced by the ruminant animal.

### ADVANTAGES

The liquid feed formulations described in embodiments of the present invention herein provide a number of advantages, including, but not limited to:
[1] Being able to maintain viscosity (flowability & pumpability) and maintain consistency (not settle out or separate) of the liquid feed formulation;
[2] Improve palatability of the Asparagopsis species; and
[3] Minimise the loss of bromoform in the liquid feed formulation.

### Embodiments:

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used.

Although the invention has been described with reference to specific examples, it will be appreciated by those skilled in the art that the invention may be embodied in many other forms.

### Industrial Applicability

It is apparent from the above, that the arrangements described are applicable to the animal feed industry.

1. A liquid feed formulation for reducing methane production by a ruminant animal, said liquid feed formulation comprising an effective amount of at least one species of red marine microalgae suspended within a liquid nutrient, said liquid feed formulation, being pumpable, having a viscosity of between 1200 cP and 2200 cP at 23° C.

2. The liquid feed formulation according to claim 1, wherein the species of red marine microalgae is an Asparagopsis spp. selected from the group consisting of Asparagopsis taxiformis and Asparagopsis armata.

3. The liquid feed formulation according to claim 1, wherein the liquid feed formulation has a dry matter content of from about 50% to about 75% by weight.

4. The liquid feed formulation according to claim 1, wherein the species of red marine microalgae is freeze dried.

5. The liquid feed formulation according to claim 1, wherein the species of red marine microalgae is homogenised in an edible oil selected from the group consisting of canola oil, sunflower oil, safflower oil, soybean oil, and any combination thereof.

6. The liquid feed formulation according to claim 1, wherein the species of red marine microalgae comprises an anti-methanogenic agent.

7. The liquid feed formulation according to claim 6, wherein the anti-methanogenic agent is bromoform.

8. The liquid feed formulation according to claim 7, wherein the bromoform is present in the liquid feed formulation in an amount from about 100 mg/kg to about 700 mg/kg by weight.

9. The liquid feed formulation according to claim 1, wherein the liquid nutrient comprises water.

10. The liquid feed formulation according to claim 9, wherein the water is present in the liquid feed formulation in an amount from about 10% to about 35% by weight.

11. The liquid feed formulation according to claim 1, wherein the liquid nutrient comprises a carbohydrate source.

12. The liquid feed formulation according to claim 11, wherein the carbohydrate source is present in the liquid feed formulation in an amount of about 20% to about 80% by weight.

13. The liquid feed formulation according to claim 11, wherein the carbohydrate source comprises a sugar-based carbohydrate source.

14. The liquid feed formulation according to claim 13, wherein the sugar-based carbohydrate source comprises molasses and/or glycerine.

15. The liquid feed formulation according claim 1, wherein the liquid nutrient comprises an edible oil selected from the group consisting of canola oil, sunflower oil, safflower oil, soybean oil, and any combination thereof.

16. The liquid feed formulation according claim 15, wherein the edible oil is present in the liquid feed formulation in an amount from about 0.01% to about 0.5% by weight.

17. The liquid feed formulation according claim 15, wherein the edible oil comprises canola oil.

18. The liquid feed formulation according claim 1, wherein the liquid nutrient comprises a thickener selected from the group consisting of xanthan gum, sodium alginate, gum arabic, sodium carboxymethylcellulose, guar gum, maltodextrin, pregelatinized starch and soy protein binder.

19. The liquid feed formulation according claim 18, wherein the thickener is present in the liquid feed formulation in an amount from about 0.01% to about 0.5% by weight.

20. The liquid feed formulation according claim 18, wherein the thickener comprises xanthan gum.

21. The liquid feed formulation according to claim 1, further comprising one or more excipients or additives selected from the group consisting of wetting agents, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen).

22. The liquid feed formulation according to claim 1, wherein the effective amount of the at least one species of red marine microalgae suspended within the liquid nutrient is from about 2% to about 15% by weight.

23. The liquid feed formulation according to claim 1, wherein the liquid nutrient comprises:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source.

24. The liquid feed formulation according to claim 23, wherein the edible oil is canola oil, the carbohydrate source is molasses, and the thickener is xanthan gum.

25. A liquid feed formulation for reducing methane production by a ruminant animal, said liquid feed formulation comprising an effective amount of at least one species of red marine microalgae suspended within a liquid nutrient comprising:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source,
wherein the liquid feed formulation is pumpable, having a viscosity of between 1200 cP and 2200 cP at 23° C.

26. The liquid feed formulation according to claim 25, wherein the species of red marine microalgae is Asparagopsis taxiformis.

27. The liquid feed formulation according to claim 26, wherein the effective amount of Asparagopsis taxiformis suspended within the liquid nutrient is from about 2% to about 15% by weight.

28. The liquid feed formulation according to claim 26, wherein the Asparagopsis taxiformis comprises an anti-methanogenic agent in the form of bromoform.

29. The liquid feed formulation according to claim 28, wherein the bromoform is present in the liquid feed formulation in an amount from about 100 mg/kg to about 700 mg/kg by weight.

30. The liquid feed formulation according to claim 25, wherein the liquid feed formulation has a dry matter content of from about 50% to about 75% by weight.

31. A method of feeding a ruminant animal, the method comprising feeding the liquid feed formulation of any one of claims 1 to 30 to a ruminant animal.

32. The method according to claim 31, wherein the liquid feed formulation is mixed with a feedstock.

33. The method according to claim 32, wherein the liquid feed formulation is mixed with the feedstock at a ratio of about 2% to about 8% relative to the combined weight of the liquid feed formulation and the feedstock.

34. The method according to claim 32 or 33, wherein the feedstock is selected from the group consisting of dry animal fodder, straw, hay, alfalfa, grains, forage, grass, fruits, vegetables, oats, crop residue, fats and oils and any combination thereof.

35. The method according to claim 31, wherein said ruminant animal is cattle, sheep or goat.

36. A method for reducing methane production by a ruminant animal, said method comprising the step of administering to said animal a liquid feed formulation comprising an effective amount of at least one species of red marine microalgae suspended within a liquid nutrient, said liquid feed formulation being pumpable, having a viscosity of between 1200 cP and 2200 cP at 23° C.

37. The method according to claim 36, further comprising the step of mixing the liquid feed formulation with a feedstock.

38. The method according to claim 37, wherein the liquid feed formulation is mixed with the feedstock at a ratio of about 2% to about 8% relative to the combined weight of the liquid feed formulation and the feedstock.

39. The method according to claim 37 or 38, wherein the feedstock is selected from the group consisting of dry animal fodder, straw, hay, alfalfa, grains, forage, grass, fruits, vegetables, oats, crop residue, fats and oils and any combination thereof.

40. The method according to claim 36, wherein said ruminant animal is cattle, sheep or goat.

41. A method of preparing a liquid feed formulation for reducing methane production by a ruminant animal, the method comprising the step of suspending an effective amount of at least one species of red marine microalgae within a liquid nutrient to form a liquid feed formulation that is pumpable, having a viscosity of between 1200 cP and 2200 cP at 23° C.

42. The method according to claim 41, wherein the species of red marine microalgae is an Asparagopsis spp. selected from the group consisting of Asparagopsis taxiformis and Asparagopsis armata.

43. The method according to claim 41, wherein the liquid feed formulation has a dry matter content of from about 50% to about 75% by weight

44. The method according to claim 41, wherein the species of red marine microalgae is freeze dried.

45. The method according to claim 44, wherein the liquid nutrient comprises:
(a) about 10% to about 35% by weight of water;
(b) about 0.01% to about 0.5% by weight of a thickener;
(c) about 0.01% to about 0.5% by weight of an edible oil; and
(d) about 20% to about 80% by weight of a carbohydrate source.

46. The method according to claim 45, wherein the water, thickener and edible oil of the liquid nutrient are combined sequentially, prior to suspending the at least one species of red marine microalgae therewithin.

47. The method according to claim 46, wherein the carbohydrate source is introduced to the liquid nutrient after the at least one species of red marine microalgae has been suspended therewithin.

48. The method according to claim 41, wherein, prior to suspending the species of red marine microalgae within the liquid nutrient, the method comprises the step of homogenising said species of red marine microalgae in an edible oil selected from the group consisting of canola oil, sunflower oil, safflower oil, soybean oil, and any combination thereof.

49. The method according to claim 48, wherein the liquid nutrient comprises:
(a) about 10% to about 35% by weight of water;
(b) about 0.01% to about 0.5% by weight of a thickener; and
(d) about 20% to about 80% by weight of a carbohydrate source.

50. The method according to claim 49, wherein the water and thickener of the liquid nutrient are combined sequentially, prior to suspending the homogenised species of red marine microalgae therewithin.

51. The method according to claim 50, wherein the carbohydrate source is introduced to the liquid nutrient after the homogenised species of red marine microalgae has been suspended therewithin.

52. The method according to claim 41, wherein the species of red marine microalgae comprises an anti-methanogenic agent.

53. The method according to claim 52, wherein the anti-methanogenic agent is bromoform.

54. The method according to claim 53, wherein the bromoform is present in the liquid feed formulation in an amount from about 100 mg/kg to about 700 mg/kg by weight.

55. The method according to claim 49, further comprising the step of introducing one or more excipients or additives to the liquid nutrient prior to suspending the at least one species of red marine microalgae therewithin.

56. The method according to claim 55, wherein the one or more excipients or additives are selected from the group consisting of wetting agents, pH modifiers, sources of amino acids, peptides, proteins, vitamins, microelements, fats, fatty acids, lipids, carbohydrates, sterols, enzymes, calcium, magnesium, phosphorus, potassium, sodium, chlorine, sulfur, chromium, cobalt, copper, iodine, iron, manganese, molybdenum, nickel, selenium, zinc, milk production enhancers, emulsifiers and nitrogen as NPN (non-protein nitrogen).

57. A liquid feed formulation for reducing methane production by a ruminant animal, the liquid feed formulation prepared according to the method of claim 41.
